(19) United States
(12) Patent Application Publication
(10) Pub. No.: US 2020/0383587 A1
(43) Pub. Date: Dec. 10, 2020

(54) EXAMINING BLOOD FLOW PATTERNS IN THE BLOOD VESSELS OF A PATIENT USING PHASE CONTRAST MRI

(71) Applicant: CHRISTIAN-ALBRECHTS-UNIVERSITÄT ZU KIEL, Kiel (DE)

(72) Inventor: Dominik Daniel GABBERT, Kiel (DE)

(21) Appl. No.: 16/631,243

(22) PCT Filed: Jul. 17, 2018
(86) PCT No.: PCT/EP2018/069396

§ 371 Date: May 29, 2020

(30) Foreign Application Priority Data
Jul. 18, 2017 (DE) 10 2017 116 144.9
Jul. 27, 2017 (DE) 10 2017 117 022.7

(51) Int. Cl. A61B 5/026; A61B 5/055; A61B 5/0285; A61B 5/00; G01R 33/563; G01R 33/56; G06T 7/00

(52) U.S. Cl. CPC A61B 5/0263; A61B 5/055; A61B 5/0285; A61B 5/7475; G01R 33/56325; G06T 2207/30048; G01R 33/56316; G06T 7/0012; G06T 2207/10088; G06T 2207/30104; G01R 33/5608

(57) ABSTRACT

The invention relates to a technique for examining blood flow patterns in the blood vessels of a patient (1), the technique having the following features: collecting raw data from a time- and space-resolved MRI phase contrast measurement of the cardiovascular system of a patient or parts thereof (1), or reading such data or data determined therefrom via an input interface (3), and calculating at least one primary variable which quantifies the blood flow pattern; carrying out multi-plane reconstructions on the basis of at least one calculated primary variable along a defined path (9) which reproduces the course of a blood vessel of the patient (1) to obtain a local distribution of the at least one primary variable in the vessel cross-section; and calculating and outputting at least one secondary variable which quantifies the blood flow pattern as a function of the position along the course of the vessel on the basis of the at least one primary variable after carrying out the multi-plane reconstructions.

The invention relates to a method of quantifying fluid dynamic properties of the blood flow of a patient through the blood vessels, as per the features of claim 1. The invention further relates to a computer program for carrying out such a method and to a device which is likewise configured to carry out such a method.

The invention builds on a specific MRI technique. The abbreviation “MRI” represents magnetic resonance imaging which is also referred to as nuclear magnetic resonance and which is used, in particular, for clinical examination of cardiovascular diseases as well. In contrast to a computed tomography examination, a variety of properties of tissue and blood can be highlighted by contrasts within the scope of an MRI examination. Inter alia, blood flows can be measured and represented by grayscale or color values. The technique for measuring blood flows is referred to as phase contrast. By now, conventional phase-contrast measurements, i.e., time-resolved 2-D phase-contrast measurements, have become part of the standard repertoire of cardiovascular MRI examinations for measuring the blood flow through a vessel. Here, the measurement is carried out in a plane which must be angled orthogonal to the orientation of the vessel (cross-sectional plane) during examination by the examiner. Here, the component of the blood flow velocity orthogonal to the plane is measured at each pixel. With the aid of analysis software, the vessel is selected in the measured plane by a contour and time-resolved, area-related quantities can be determined. Area-related quantities are understood to mean quantities whose ascertainment requires a mathematical operation over an area. Typical examples of this include the flow, which specifies the volume of blood per unit time that flows through the vessel cross section, mean blood flow velocity, maximum velocity within the cross-sectional plane at any time and eccentricity.

A known extension of the phase-contrast technique facilitates the time- and space-resolved (“4D flow”) measurement of blood flow velocities v in a volume, which is decomposed into three-dimensional pixels (voxels). This extension yields the additional option of characterizing complex flow patterns and quantifying these by way of flow quantities. Here, the term “complex” does not mean the mathematical term but instead, as a distinction from the area-related quantities, the less elementary property of flow patterns that may be exhibited when observing the spatial flow vector in a volume. Therefore, complex flow quantities are understood to mean quantities that characterize the blood flow, the calculation of which requires a mathematical operation over a volume and/or the calculation of which depends on all three spatial directions. Moreover, area-related quantities can also be obtained from 4D flow measurements. All variables specified within the scope of this invention are defined in table 1.

Extending the phase-contrast technique to 4D flow and the increase in potential variables that can be used to characterize the blood flow, accompanying this, has created the need for a systematic quantification, which has not been covered by any known technique. For instance, the question whether, and to what extent, turbulences increase or decrease at what longitudinal position along the vessel course, which arises when assessing surgical corrections or for the planning thereof, currently cannot be satisfactorily answered. The clinical significance of turbulences is evident from numerous studies. Formulas for calculating turbulent energy and turbulent energy density of individual voxels were proposed and have in the meantime been comprehensively validated in studies on the basis of 4D flow measurements, but what still is missing today is a systematic, statistically evaluable process for quantifying turbulences in the vessel course. Similarly, the options of using other complex flow patterns for a fluid dynamic understanding of the relevant patient, e.g., the strength of the helical flow profile, which can be quantified by helicity, has not yet been fully exploited. This is because, to date, no method has arisen either for determining the helicity, which method makes do without an arbitrary prescription of usually large vessel sections and supplies a systematic, highly resolved quantification in the vessel course with the aid of multiplanar reconstructions.

The invention is based on the object of exploiting the quantification potentials of raw data captured by means of 4D phase-contrast MRI measurements to form a more comprehensive but, in the process, uniform fluid dynamic assessment of blood vessels and to develop new possibilities for clinical diagnosis.

According to claim 1, this object is achieved by a method for determining fluid dynamic flow quantities of the blood flow along the course of a blood vessel of a patient, including the following features:
a) capturing the raw data, i.e., data that have not yet been interpreted in respect of the desired contrast, of a time- and space-resolved MRI phase-contrast measurement (measurement of blood flow patterns in three-dimensional space) of the cardiovascular system of a patient or parts thereof (1), or reading such raw data and/or data, which reproduce contrasts, determined therefrom by way of an input interface (3), and calculating at least one primary quantity that quantifies the blood flow pattern;
b) performing multiplanar reconstructions along a set path on the basis of the at least one primary quantity calculated in method step (b), said path reproducing the course of a blood vessel of the patient, in order to obtain a spatial distribution of the at least one primary quantity in the vessel cross section;
c) calculating and outputting at least one secondary quantity of the blood flow, which quantifies the blood flow pattern, as a function of the position along the vessel course on the basis of the at least one primary quantity following the performance of the multiplanar reconstructions.

According to the invention, multiplanar reconstructions are carried out on the basis of at least one primary quantity, obtained in method step (b), along a set path, and hence not in conventional fashion on the basis of material or tissue properties, such as, e.g., the absorption spectrum in the case of computed tomography or signal decay times in MRI.

Using simple and cost-effective means, the invention allows a significant extension of the examination possibilities of MRI and allows improved diagnoses. This is facilitated by virtue of the MRI data, which is available in any case, or data determined therefrom being processed further as specified above in order to ascertain therefrom fluid dynamic flow quantities in statistical and clinically evaluable form, i.e., as a function of the longitudinal position along the vessel course and the time corresponding to claim 2. This allows precious and clinically highly relevant parameters to be taken into account, which significantly improves the possibilities of making diagnoses and planning and assessing surgical interventions. What should be highlighted in particular is that the invention reduces the amount of work required for evaluating fluid dynamic quantities since a large number of fluid dynamic flow quantities can be calculated in parallel. In parallel means that none of the calculations for these flow quantities require specific user prescriptions and all flow quantities can be calculated uniformly in a workflow on the basis of general user prescriptions.

Multiplanar reconstruction, also referred to as multiplanar reformatting or abbreviated MPR, is a method for a two-dimensional image reconstruction in planes. The position of such a plane is predetermined with any alignment and the image data, which are measured in the three-dimensional pixels (voxels) in the surroundings of the plane, are projected onto the plane. Using this technique, it is possible to convert the originally measured image stack with the alignment predetermined by the measurement into image stacks with any new alignment. Traditionally, this technique is used in CT examinations. It requires the one-time measurement of a volume with a good spatial resolution in any alignment of the image stack and allows the calculation of a new image stack with any other alignment on the basis of this measurement. By way of example, the measurement of the layers along the body axis (axial image stack) of a patient suffices to calculate coronal (approximately parallel to the abdominal wall, view from the front on the patient) or sagittal image stacks (view from the side on the patient), or to calculate any other image stack. Multiplanar reconstructions can be performed along one axis or along a curved path. The planes are each oriented orthogonal to the axis or the curved path. Here, the set path can be predetermined manually by the user, for example, or else it can be calculated automatically according to predetermined criteria. Multiplanar reconstructions are usually applied to the measured image contrasts representing material or tissue properties.

The technique of multiplanar reconstruction is used in a novel way within this invention, specifically, and for the first time, on the basis of selected primary quantities to quantify the blood flow pattern, which quantities are calculated voxel-by-voxel in advance for the purposes of quantifying vortices and which quantities, following the multiplanar reconstructions , serve to calculate the secondary flow quantities specified in claims 5 and 7: the vorticity, which quantifies the strength of vortices, the dimensions of which are greater than the order of magnitude of a voxel, and the turbulent kinetic energy density, which quantifies the energy density of vortices within a voxel that is dissipated into heat and lost from the flow energy at the end of a cascade, and optionally further (voxel-by-voxel) quantities. The multiplanar reconstructions are carried out along the vessel course and initially supply the values of the primary quantities in the vessel cross section at each position of the vessel course. According to claim 3, the multiplanar reconstructions are carried out continuously. The term “continuously” denotes a spatial spacing of the multiplanar reconstructions along the path (central line), which is chosen to be small enough to capture the fluid dynamic profile in the vessel. Since the voxel size of the measurement is often also selected according to this criterion (usually of the order of 1.5-2.5 mm), the distances in this case should be selected in the order of the voxel size. Smaller distances do not increase information content of the result. The term “continuously” also highlights the automatic nature of the process and the complete determination, as opposed to sampling, of flow variables and it serves to delimit from the manual prescription of individual positions.

By continuously carrying out the multiplanar reconstructions over the entire vessel, or at least a portion of the vessel, it is possible to quantify medically relevant properties of the blood flow, which may be overlooked when setting measurement positions manually. By way of example, what may arise when positions are set manually is that it is not the maximum or minimum values of the quantities to be determined that are captured. Such disadvantages are avoided by continuously carrying out the multiplanar reconstructions along the set path of the vessel.

The path for calculating the multiplanar reconstructions along the course of a vessel is represented by a central line, which is determined by virtue of
a) manually set position marks in the center of the respective vessel cross section being interconnected by a smooth curve. or
b) semiautomatic or automatic processes of central line detection being used.

After carrying out the multiplanar reconstructions according to method step b of claim 1, the primary fluid dynamic quantities (e.g., according to claims 4 and 6, the vorticity, the turbulent kinetic energy density and the velocity) are initially available in the cross-sectional planes and these serve in method step c of claim 1 to calculate secondary fluid dynamic quantities. According to claim 6, the multiplanar reconstructions are performed once for all primary quantities in a multidimensional space. The multi-dimensional space emerges from the primary quantities. “Once for all primary quantities” means that the multiplanar reconstructions are not carried out separately for each of the primary quantities but are carried out in a single computational process in order to save computation time. Automatic and/or semiautomatic segmentation techniques can be used to delimit the vessel volume from the surrounding tissue and restrict the calculation to the vessel volume. As a result of an embodiment of the invention developed thus, it is possible to calculate all fluid dynamic quantities specified in claim 7 in parallel and, as a result, the following are available systematically as a function of the longitudinal position along the vessel course and, where appropriate, as a function of time in accordance with claim 2: the turbulent kinetic energy density related to the vessel cross section or a corresponding layer (e.g., obtained by averaging), turbulent kinetic energy, root mean square turbulent kinetic energy density, helicity density, helicity, relative helicity density, circulation rate, mean vorticity, mean flow velocity, maximum flow velocity, blood flow (volume per unit time), eccentricity, local radii of curvature of the vessel course, local torsion of the vessel course, size of the vessel cross section (i.e., area and/or equivalent diameter and/or equivalent radius).

Moreover, further derived quantities such as maximum values, minimum values, standard deviation and further quantities emerge from the distribution of the fluid dynamic quantities in the vessel cross section. On account of the elementary character of the primary quantities calculated in method step (a) of claim 1 and planar-reconstructed in method step (b) of claim 1, this process can be used to introduce and examine any number of new secondary fluid dynamic quantities in order to quantify observed flow patterns. Secondary quantities that require a mathematical operation over volume (e.g., helicity) can be determined just like quantities that require a mathematical operation over an area (such as, e.g., circulation rate), even though planes, i.e., areas, are determined during the multiplanar reconstruction. Since the multiplanar reconstructions within the scope of the application according to the invention are carried out at small distances, the distances from one another should be interpreted as layer thicknesses. This procedure allows a systematic consideration of quantities with different dimensionality.

If the vessel segmentation and the central line are provided for performing the multiplanar reconstructions according to the invention, it is possible to calculate geometric parameters such as, e.g., the cross-sectional area, equivalent diameter, curvature, torsion, and effective torsion (product of curvature and torsion). No further prescriptions are necessary. The geometric properties of the vessel course (local radius of curvature, local torsion, local effective torsion) can be calculated from the central line. The geometric properties of the vessel size (cross-sectional area, equivalent diameter) can be calculated from the vessel segmentation. The flow dynamics are related to the geometric properties of the vessel. The invention allows quantifying these relationships without further user inputs.

The classification of the available results is suitable for the clinical-statistical evaluation of the flow dynamics.

The distortion of the image stack following the performance of the MPR, which is caused by the curvature of the vessel, for example prevents the vorticity according to the formula specified below from being able to be readily calculated from the velocity. If a continuous multiplanar reconstruction were to be only applied to velocities as primary quantity, secondary flow quantities that depend on the rotation of the velocity field ∇×v (mean vorticity, circulation rate, helicity density, helicity, relative helicity density) could not be calculated from the formulas specified below on account of the curvature-related distortion in step c of claim 1. Therefore, the vorticity is calculated voxel-by-voxel, the MPR is subsequently performed and then the secondary flow quantities with respect to the plane or layer are calculated therefrom.

According to an advantageous embodiment, provision is made for a curved multiplanar reconstruction of quantities characterizing the flow dynamics to be performed along the central line in the vessel (MPR path). This can avoid a fixed alignment and/or fixed angling of planes of the multiplanar reconstruction having to be provided. Moreover, the multiplanar reconstruction can be carried out fully automatically because it is not necessary to position and/or angle planes of the multiplanar reconstruction in manual fashion. In this way, the multiplanar reconstruction (MPR) can automatically adapt to the course of the vessel.

Such MPRs along a path, which are also referred to as curved MPRs, have previously not been applied to fluid dynamic quantities from MRI measurements. Manual positioning and/or angling, as is evidently carried out in [1] without the use of an automatic technique, are time-consuming and imprecise. In [1], MRA (magnetic resonance angiography) is visually used to align the planes: “PC-MRA was used for anatomic orientation in 30 (EnSight, CEI, USA) and to position equally spaced (distance 10 mm) analysis planes along the entire thoracic aorta” The use of a specific technique is not specified here. The invention present here facilitates a simultaneously well-resolved and time-efficient fluid dynamic analysis along the course of a vessel without further manual angling of individual planes. The spacing of the MPR planes can automatically be chosen to be small without greater amounts of work so as to achieve a high resolution.

According to an advantageous embodiment, the invention includes combining various quantities, which characterize the flow dynamics and are at least partly complementary, to form a multidimensional space, on the basis of which the multiplanar reconstruction is carried out. The flow dynamic complexity in vessels was not previously systematically mapped by any known technique. By combining different, partly complementary fluid dynamic quantities (e.g., velocity, vorticity, turbulent kinetic energy density) to form a multidimensional fluid dynamic image space, the invention present here facilitates the comprehensive determination of secondary quantities as a function of the vessel position, which reflects the flow behavior to an extent not achieved previously. The combination of magnitude and flow velocity, used in various publications, should not be understood as one of those described above since the magnitude image only serves to present anatomical conditions and forms the basis for calculating a PC-MRA. By way of example, complementary quantities are the (mean) velocity, the vorticity and the turbulent kinetic energy density. In turbulence theory of fluid dynamics, the Reynolds decomposition is understood to mean the decomposition of the velocity field into a mean velocity field and a fluctuating velocity field. A PC-MRI measurement can be understood to be a realization of such a Reynolds decomposition. It supplies two complementary quantities: a mean velocity field and the voxel-related standard deviation (intravoxel standard deviation, IVSD), from which the turbulent kinetic energy can be calculated. While mean velocity and turbulent kinetic energy density are quantities that relate to properties of the flow dynamics within an individual voxel, the vorticity quantifies the vortex formation at an order of magnitude above the voxel size, and is consequently also complementary.

According to an advantageous embodiment, provision is made for the central line in the vessel to be used together
a) for performing the multiplanar reconstruction for the purposes of determining secondary fluid dynamic quantities along the vessel and
b) for determining geometric properties such as curvature and torsion of the blood flow and/or of the vessel.

Using geometric properties of an MPR path has not been proposed in any known technique. Thus, the path (central line) in the present invention is both the initial point of the MPR and the source of geometric information such as curvature and torsion, which, according to the invention, are brought into similar form as secondary flow quantities (function of the position along the vessel course) and can be compared to the flow quantities. This should be highlighted in particular for the reason that the flow dynamics of vessels greatly depend on geometric properties of the vessel and a suitable assessment of cause and effect of observed phenomena is only ensured by the completeness of the data evaluation with the aid of the invention.

The method according to the invention can also be used in existing MRI installations, e.g., by a software extension or by way of an additional computing unit. As a result of this, the implementation outlay for the invention is low. Retrofitting of existing installations is readily possible. An offline evaluation of the data obtained by the MRI measurements, independently of the time of the MRI measurements, is also readily possible.

Consequently, the invention supplies a technically easily realizable solution for a uniform, systematic, comprehensive determination of all relevant flow quantities in a single workflow, for the purposes of a clinical assessment.

Describing the flow dynamics by way of individual quantities can be considered to be a simplification of a complex structure. However, collecting simple quantities for the purposes of statistical analysis for setting criteria for clinical decisions has proven its worth in medicine. In order to satisfy the demand for completeness as comprehensively as possible at the same time, the number of considered quantities and the employed spatial resolution of the quantities is necessarily high. This provides a motivation for the described invention.

The object specified at the outset is further achieved by a computer program comprising program code means that are configured to carry out the method as described above when the computer program is executed on a computer. By way of example, the computer can be a microprocessor, a commercially available computer, a computing unit of an MRI installation or any other type of computer that is suitable for executing computer programs. The above-described advantages can also be realized hereby.

The object specified in the outset is further achieved by a device for determining complex fluid dynamic flow quantities of the blood flow in a blood vessel, comprising the following features:
a) an input interface that is configured to read raw data of a time- and space-resolved MRI phase-contrast measurement of the cardiovascular system of a patient or parts thereof, or data determined therefrom,
b) a computing unit that is configured to carry out the method as described above,
c) an output interface for outputting at least one complex fluid dynamic flow quantity of the blood flow through the vessel on the basis of the results of the continuously performed multiplanar reconstructions.

The above-described advantages can also be realized hereby. The invention may comprise an MRI measuring device that is coupled to the input interface. In this way, the raw data captured by the MRI measurements or data determined therefrom can be supplied directly to the computing unit via the input interface. The computing unit can be a computing unit of an MRI installation which also comprises the MRI measuring device. The computing device can also be a separate computing device, which is advantageous, in particular, within the scope of the offline evaluation of the data obtained by the MRI measurements.

The output of the at least one complex fluid dynamic flow quantity can be an output on another appliance, e.g., a graphical output on an image display appliance, or an output to any other appliance by way of any output interface, for example for further processing of the output quantity. The output can also be an internal output of the quantity to another component of a computing unit or to another software module, in which the quantity is processed further.

The invention is explained in more detail below on the basis of exemplary embodiments, with use being made of the drawings.

In the drawings:

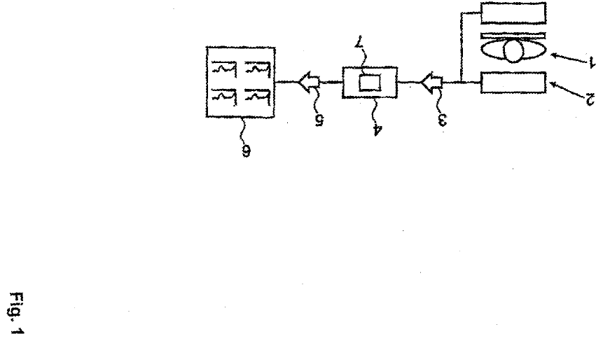

FIG. 1 shows a device for determining complex fluid dynamic flow variables;

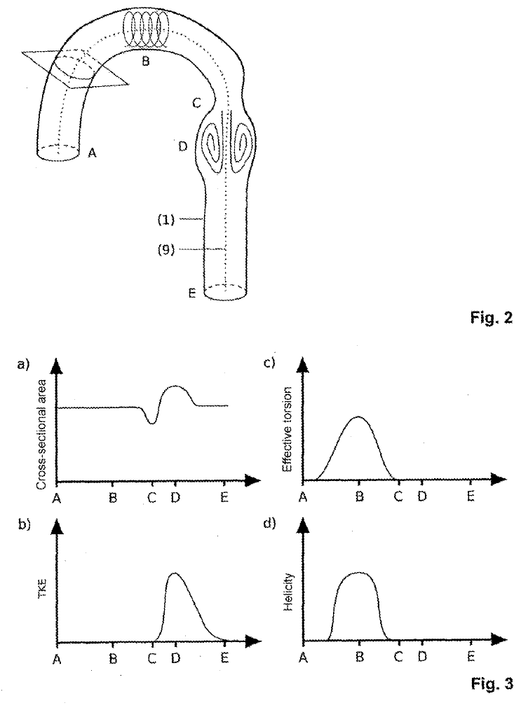

FIG. 2 shows a schematic illustration of a blood vessel with geometric and fluid dynamic peculiarities;

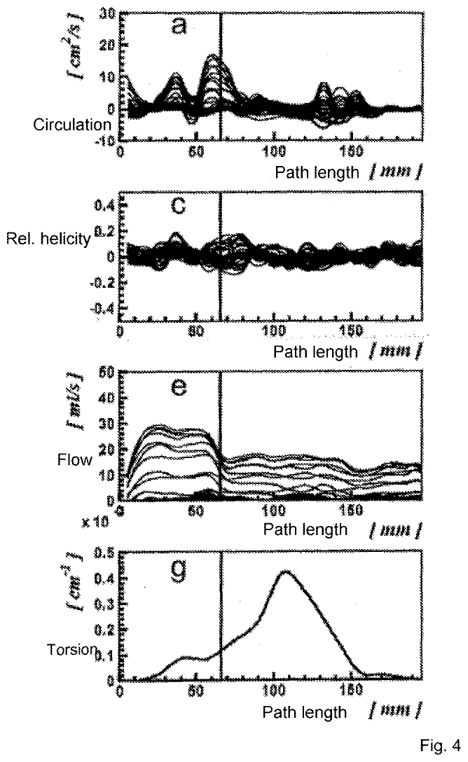

FIG. 3 shows the schematic illustration of four secondary quantities obtained from FIG. 2; and

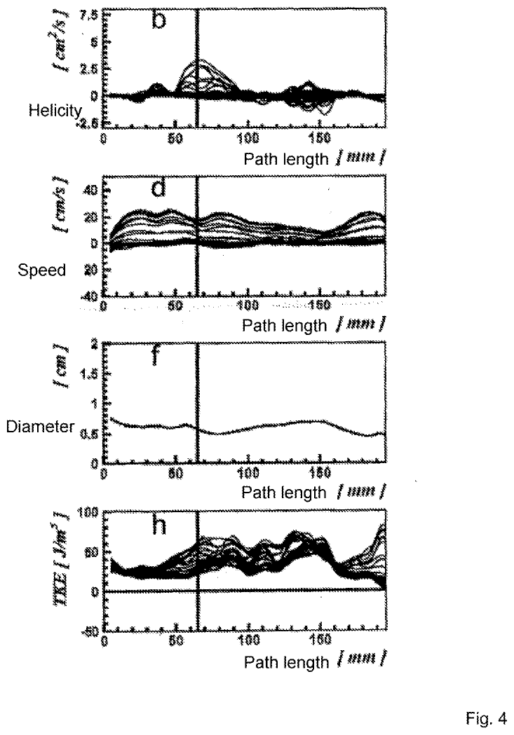

FIG. 4 shows further graphical representations of secondary quantities.

The device for determining complex fluid dynamic flow quantities, represented in a schematic illustration in FIG. 1, comprises a computer device 4 with a computing unit 7 (processor unit). The computer device 4 further comprises a memory or is connected to a memory, in which a computer program for executing the method according to the invention is stored. The method is executed by the computing unit 7. The computing device 4 comprises an input interface 3, by means of which the computing device 4 is coupled to an MRI installation or, at least, to an MRI measuring device 2. By means of the MRI measuring device 2, it is possible to capture body-internal data of a patient 1 by way of magnetic resonance imaging, for example the vessels of said patient and fluids flowing therein. The computing device 4 further comprises an output interface 5, to which further appliances can be connected. What is illustrated in exemplary fashion is that a display device 6, for example in the form of an image display device, is connected to the output interface 5. The complex fluid dynamic flow quantities ascertained by the computing device 4 and further quantities can be visually output, for example in the form of diagrams, on the display unit 6. Examples of image representations on the image display appliance 6 are shown in FIGS. 2 to 4.

For elucidation purposes, a vessel portion of the patient 1 is schematically illustrated in FIG. 2, said vessel portion extending from point A to point E. The dotted line 9 is the central line of the vessel. As an example of the continuously performed multiplanar reconstructions, a plane aligned orthogonal to the central line 9 is illustrated. The vessel course is curved in the surroundings of the position B. The vessel is constricted at position C and dilated at position D. The blood flow exhibits a helical pattern at position B. The blood flow exhibits turbulences at position D.

FIG. 3 schematically illustrates, by way of example, selected results of fluid dynamic and geometric quantities, which can be collected by the computing device for the case illustrated in FIG. 2 by means of the method according to the invention. The longitudinal vessel position is plotted along the abscissa. The cross-sectional area is plotted on the ordinate in diagram (a) and the turbulent kinetic energy density (TKE) is plotted on the ordinate in diagram (b). High turbulent energy means a loss of kinetic flow energy. The effective torsion (product of curvature and torsion) is plotted on the ordinate in diagram (c) and the helicity is plotted in diagram (d). A high helicity describes flow patterns with great rotation and forward motion. The helicity illustrated in diagram (d) is positive, i.e., the helical flow pattern corresponds to the winding of a right-handed screw. The classification of process and presentation of the results allows statistically evaluable relationships between vessel geometry and flow dynamics to be uncovered and quantified.

A plurality of quantities determined from the MRI data can also be output simultaneously by the computing unit 4, as shown in FIG. 4. There, the circulation of the blood flow in the vessel is illustrated in diagram (a), the helicity of the blood flow is illustrated in diagram (b), the relative helicity density of the blood flow is illustrated in diagram (c), the speed of the blood flow is illustrated in diagram (d), the flow rate of the blood flow is illustrated in diagram (e), the vessel diameter is illustrated in diagram (f), the torsion of the vessel is illustrated in diagram (g) and the turbulent kinetic energy of the blood flow is illustrated in diagram (h), in each case over the spatial coordinate in the vessel 8, which has already been mentioned with reference to FIG. 3. Each of the flow quantities was measured in 24 equidistant phases within the cardiac cycle. Each of the diagrams plots the curves of all phases in distinguishable fashion. The described invention supplies, with a few user specifications, clinically relevant, highly resolved information for the comprehensive and, at the same time, simple and systematic examination of the blood flow.

[1] Lorenz, R. [et al]: 4D flow magnetic resonance imaging in bicuspid aortic valve disease demonstrates altered distribution of aortic blood flow helicity. In: Magnetic resonance in medicine, vol. 71, 2014, no. 4, pp. 1542-1553

TABLE 1
Variables and formulas

| | | |
|---|---|---|
| i | = 1, . . . , n | Pixels of the vessel cross section |
| j | = 1, . . . , 3 | Spatial directions |
| rᵢ | | Position vectors of the pixels |
| v | | Velocity |
| ν | | Speed |
| ν¹ | | Orthogonal velocity component |
| A | | Cross-sectional area |
| ρ | | Density of the fluid |
| ν_{enc}^{j} | | Velocity encoding in direction j |
| \|S\| | | Signal magnitude without first-order |
| | | velocity encoding |
| \|Sⱼ\| | | Signal magnitude with velocity |
| | | encoding in direction j |
| ω | = ∇ × v | Vorticity |
| Γ | $= {\int }_{A} ω dA$ | Circulation rate |
| ω̅ | $= \frac{{\int }_{A} ω d A }{A}$ | Mean vorticity |
| H_{d} | = v · ω | Helicity density |
| H | $= {\int }_{A} v \cdot ω dA$ | Helicity |
| Hᵣ | $= \frac{v \cdot ω}{\vert v \vert \cdot \vert ω \vert}$ | Relative helicity density |
| E | $= \frac{{\sum }_{i} {r}_{i} \vert {v}_{i} \vert}{{\sum }_{i} \vert {v}_{i} \vert} - \frac{{\sum }_{i} {r}_{i}}{n}$ | Eccentricity |
| TKE | $= ρ {\sum }_{ji = 1}^{a} {v}_{enc}^{ij} ln ( \frac{\vert S \vert}{\vert {S}_{i} \vert} )$ | Turbulent kinetic energy density [1] |

[1] Dyverfeldt, P., Sigfridsson, A., Kvitting, J.-P. E. and Ebbers, T. (2006), Quantification of intravoxel velocity standard deviation and turbulence intensity by generalizing phase-contrast MRI. Magn. Reson. Med., 56: 850-858. doi:10.1002/mrm.21022

1. A method for examining blood flow patterns in a blood vessel of a patient, comprising:
a) capturing raw data of a time- and space-resolved MRI phase-contrast measurement of a cardiovascular system of a patient or parts thereof, or reading the raw data and/or data determined therefrom by way of an input interface, and calculating at least one primary quantity that quantifies a the blood flow pattern;
b) performing multiplanar reconstructions along a set path on a basis of the at least one primary quantity calculated in method step (a), said set path reproducing a course of a blood vessel of the patient, to obtain a spatial distribution of the at least one primary quantity in a vessel cross section of the blood vessel;
c) calculating and outputting at least one secondary quantity which quantifies the blood flow pattern as a function of position along the course of the blood vessel on the basis of the at least one primary quantity following the performance of the multiplanar reconstructions.

2. The method as claimed in claim 1, wherein the at least one secondary quantity includes a plurality of secondary quantities each of which are output as a function of time and position along the course of the blood vessel.

3. The method as claimed in claim 1 wherein the multiplanar reconstructions are performed continuously along the set path.

4. The method as claimed in claim 1 wherein the at least one primary quantity includes a plurality of primary quantities, and wherein one of the primary quantities is vorticity (ω) or turbulent kinetic energy density (TKE).

5. The method as claimed in claim 1 further comprising determining one or more secondary complex quantities which quantify the blood flow pattern as a function of the position along the course of the blood vessel on a basis of the at least one primary quantity following the performance of the multiplanar reconstruction and outputting the one or more secondary complex quantities, wherein the one or more secondary complex quantities are selected from the group consisting of turbulent kinetic energy density related to the vessel cross section or a corresponding layer, turbulent kinetic energy, root mean square turbulent kinetic energy, helicity density, helicity, and relative helicity density.

6. The method as claimed in claim 1 wherein the multiplanar reconstructions of at least two primary quantities are performed once for all primary quantities in a multidimensional space.

7. The method as claimed in claim 1 wherein at least two of the following secondary quantities are determined, to be precise in parallel, on the basis of the at least one primary quantity following the performance of the multiplanar reconstruction and where at least one of the at least two secondary quantities is selected from the group consisting of turbulent kinetic energy density related to the vessel cross section or a corresponding layer, turbulent kinetic energy, root mean square turbulent kinetic energy density, helicity density, helicity, relative helicity density, circulation rate, mean vorticity, mean flow velocity, maximum flow velocity, blood flow (volume per unit time), eccentricity, and, where at least one of the at least two secondary quantities is selected from a relation to the position along the course of the blood vessel, a local radii of curvature of the course of the blood vessel, local torsion of the course of the blood vessel, and size of the vessel cross section.

8. The method as claimed in claim 1 wherein the course of the blood vessel in the body of the patient is represented by a central line which is determined by either
a) manually set position marks in a the-center of the respective vessel cross section being interconnected by a smooth curve or
b) semiautomatic or automatic processes of central line detection.

9. The method as claimed in claim 8, wherein a plane of capture of three-dimensional raw data by MRI measurements is defined at each point of the central line, said plane of capture being disposed in orthogonal fashion on the central line in each case.

10. The method as claimed in claim 1 wherein the vessel is selected by an automatic or semiautomatic segmentation technique and the calculation in method step (c) is restricted to a corresponding selection.

11. A computer program product comprising computer executable instructions encoded on a non-transient medium, wherein said instructions, when executed by a computer configured to perform the method as claimed in claim 1.

12. A device for determining complex quantities, and quantifying a blood flow pattern of blood flowing in a vessel of a patient, comprising:
a) an input interface configured to read raw data of a time- and space-resolved MRI phase-contrast measurement of a cardiovascular system of the patient or parts thereof, or data determined therefrom,
b) a computing unit configured to carry out the method as claimed in claim 1,
c) an output interface configured to output at least one complex quantity, quantifying the blood flow pattern, of the blood flow through the vessel on the basis of results of the continuously performed multiplanar reconstructions.

13. The device as claimed in claim 12, wherein the device comprises an MRI measuring device coupled to the input interface
